SEQUENCE LISTING (3 sequences)

SEQ ID NO: 1 - protein, 20 aa, Artificial Sequence
  Fully synthetic peptide
FPRPGGGGNGDFEEIPEEYL

SEQ ID NO: 2 - protein, 20 aa, Artificial Sequence
  Fully synthetic peptide
FPRPGGGGNGDFEEIPEEYL

SEQ ID NO: 3 - protein, 20 aa, Artificial Sequence
  Fully synthetic peptide
FPRPGGGGNGDFEEIPEEYL

(19) United States
(12) Patent Application Publication
(10) Pub. No.: US 2014/0187745 A1
(43) Pub. Date: Jul. 3, 2014

(54) METHOD FOR PREPARING BIVALIRUDIN

(71) Applicant: Chengdu Shengnuo Tech Co., Ltd., Chengdu (CN)

(72) Inventors: Yongjun WEN, Chengdu (CN); Qilin XIE, Chengdu (CN); Xiaoli WANG, Chengdu (CN); Dewen GUO, Chengdu (CN); Dezhi ZENG, Chengdu (CN)

(73) Assignee: Chengdu Shengnuo Tech Co., Ltd., Chengdu (CN)

(21) Appl. No.: 14/135,608

(22) Filed: Dec. 20, 2013

Related U.S. Application Data
(63) Continuation-in-part of application No. PCT/CN2011/081306, filed on Oct. 26, 2011.

(30) Foreign Application Priority Data
Jun. 23, 2011 (CN) 201110170669.1

(51) Int. Cl. C07K 7/08

(52) U.S. Cl. CPC C07K 7/08
USPC 530/326

(57) ABSTRACT

A method for preparing bivalirudin. The method includes preparing a bivalirudin resin by a solid phase synthesis, performing acidolysis of the bivalirudin resin to obtain crude bivalirudin, and purifying the crude bivalirudin to obtain purified bivalirudin. The solid phase synthesis method includes successively coupling Fmoc-protected amino acids corresponding to a sequence represented by SEQ. ID NO. 2 on an Fmoc-Leu-carrier resin through solid phase coupling synthesis to obtain the bivalirudin resin represented by SEQ. ID NO. 2.

### CROSS-REFERENCE TO RELATED APPLICATIONS

This application is a continuation-in-part of International Patent Application No. PCT/CN2011/081306 with an international filing date of Oct. 26, 2011, designating the United States, now pending, and further claims priority benefits to Chinese Patent Application No. 201110170669.1 filed Jun. 23, 2011. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 14781 Memorial Drive, Suite 1319, Houston, Tex. 77079.

### BACKGROUND OF THE INVENTION

1. Field of the Invention

The invention relates to a method for preparing bivalirudin.

2. Description of the Related Art

Bivalirudin has the structure represented by SEQ. ID NO. 1:

SEQ. ID NO. 1
D-Phe-Pro-Arg-Pro-Gly-Gly-Gly-Gly-Asn-Gly-
Asp-Phe-Glu-Glu-Ile-Pro-Glu-Glu-Tyr-Leu-OH

A large number of reports on preparation methods of bivalirudin have been disclosed both at home and abroad. All these methods employ Fmoc-Cl as an amino protective group for coupling amino acids. The structure of bivalirudin contains the Gly-Gly-Gly-Gly fragment, and thus, during successively coupling Fmoc-Gly by a solid phase method, the resulting product includes the following impurities due to the characteristics of Gly: [+1Gly]-bivalirudin, [+2Gly]-bivalirudin, [−1Gly]-bivalirudin, and [−2Gly]-bivalirudin. The impurities have similar polarity to bivalirudin, which results in the difficulty for purification of bivalirudin, thereby reducing the total yield and the product purity, and affecting medication safety.

### SUMMARY OF THE INVENTION

In view of the above-described problems, it is one objective of the invention to provide a method for producing bivalirudin. The method uses a fragment of protected amino acid Fmoc-Gly-Gly-Gly-Gly-OH, avoids the generation of impurities comprising [+1Gly]-bivalirudin, [−1Gly]-bivalirudin, [+2Gly]-bivalirudin and [−2Gly]-bivalirudin, improves the product yield and purity, presents high reaction efficiency, and is conducive to realization of a large-scale solid-phase synthesis process.

To achieve the above objective, in accordance with one embodiment of the invention, there is provided a method for producing bivalirudin. The method comprises preparing a bivalirudin resin by a solid phase synthesis method, performing acidolysis of the bivalirudin resin to obtain crude bivalirudin, and purifying the crude bivalirudin to obtain purified bivalirudin, wherein the solid phase synthesis method comprises successively coupling Fmoc-protected amino acids corresponding to a sequence represented by SEQ. ID NO. 2 on a Fmoc-Leu-carrier resin through a solid phase coupling synthesis method to obtain the bivalirudin resin represented by SEQ. ID NO. 2:

SEQ. ID NO. 2
R₁-D-Phe-Pro-Arg(Pbf)-Pro-X-Asn(R₂)-Gly-Asp(OtBu)-
Phe-Glu(OtBu)-Glu(OtBu)-Ile-Pro-Glu(OtBu)-
Glu(OtBu)-Tyr(tBu)-Leu-resin

wherein X represents Gly-Gly-Gly-Gly, R₁ represents R₃ or H, R₂ represents Trt or H, and R₃ represents Fmoc.

Solid phase coupling synthetic reaction is carried out only once to couple an X fragment, with corresponding Fmoc-protected amino acid being Fmoc-Gly-Gly-Gly-Gly-OH.

In a class of this embodiment, the Fmoc-protected amino acids comprise R₃-D-Phe-OH, Fmoc-X—OH, Fmoc-Asn(R₂)-OH, Fmoc-Arg(pbf)-OH, Fmoc-Asp(OtBu)-OH, Fmoc-Glu(OtBu)-OH, Fmoc-Gly-OH, Fmoc-Ile-OH, Fmoc-Leu-OH, Fmoc-Phe-OH, Fmoc-Pro-OH and Fmoc-Tyr(tBu)-OH., wherein X represents Gly-Gly-Gly-Gly, R₂ represents Trt or H, and R₃ represents Fmoc.

Fmoc represents 9-fluorenylmethoxycarbonyl, tBu represents tertiary butyl, Trt represents triphenylmethane, OtBu represents tert-butoxy, and Boc represents tertiary butyloxycarbonyl.

The dosage of the Fmoc-protected amino acid is between 1.2 and 6 times the total mole number of the fed resin, preferably 3 times.

Fmoc-Gly-OH is used to couple the 11^{th} Gly amino acid, and Fmoc-Gly-Gly-Gly-Gly-OH is used to couple the 13^{th} -17^{th} Gly amino acids.

The structure of Fmoc-Gly-Gly-Gly-Gly-OH is represented by Formula I:

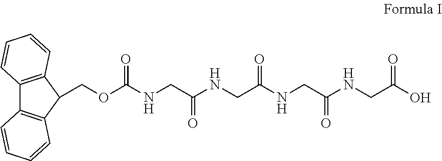 [SMILES: O=C(O)CNC(=O)CNC(=O)CNC(=O)CNC(=O)OCC1c2ccccc2-c2ccccc21]

According to the invention, a fragment of the protected amino acid Fmoc-Gly-Gly-Gly-Gly-OH is directly used to prepare bivalirudin, with the purity exceeding 99.5% and the single impurity less than 0.2%. Compared with the prior art, the method is characterized by simple reaction operation and mild reaction conditions, thus having extensive practical value and application prospect.

### DETAILED DESCRIPTION OF THE EMBODIMENTS

For further illustrating the invention, experiments detailing a method for preparing bivalirudin are described below. It should be noted that the following examples are intended to describe and not to limit the invention.

The method for preparing bivalirudin of the invention comprises preparation of a bivalirudin resin by a solid phase polypeptide synthesis method, acidolysis of the bivalirudin resin to obtain crude bivalirudin, and purification of the crude bivalirudin to obtain purified bivalirudin. The method for preparing bivalirudin resin by the solid phase polypeptide synthesis method comprises successively coupling Fmoc-protected amino acids corresponding to a sequence represented by SEQ. ID NO. 2 on a Fmoc-Leu-carrier resin through a solid phase coupling synthesis method to obtain the bivalirudin resin represented by SEQ. ID NO. 2:

SEQ. ID NO. 2
R₁-D-Phe-Pro-Arg(Pbf)-Pro-X-Asn(R₂)-Gly-Asp(OtBu)-
Phe-Glu(OtBu)-G lu(OtBu)-Ile-Pro-Glu(OtBu)-
Glu(OtBu)-Tyr(tBu)-Leu-resin

wherein X represents Gly-Gly-Gly-Gly, R₁ represents R₃ or H, R₂ represents Trt or H, and R₃ represents Fmoc.

The solid phase coupling synthesis is as follows: the protected amino acid-resin obtained by the previous reaction is subject to Fmoc deprotection and participates in the coupling reaction with the next protected amino acid. The Fmoc deprotection reagent is 10-30% (V/V) piperidine (PIP)/N,N-dimethylformamide (DMF) solution, preferably 20%. In 1 g fed resin, the dosage of the deprotection reagent is 5-15 mL, preferably 10 mL. The deprotection reaction time is 10-60 min, preferably 15-25 min

A condensing reagent and activating reagent are added during the coupling. The condensing reagent is selected from N,N-diisopropylcarbodiimide (DIC), N,N-dicyclohexylcarbodiimide (DCC), benzotriazole-1-yl-oxy-tris-pyrrolidino-phosphonium hexafluorophosphate (PyBOP), 2-(7-aza-1H-benzotriazole-1-yl)-1,1,3,3-tetramethyluronium hexafluorophosphate (HATU), benzotriazole-N,N,N′,N′-tetramethyluroniumhexafluophosphate (HBTU) or O-benzotriazole-N,N,N′,N′-tetramethyluronium tetrafluobate (TBTU), preferably N,N-diisopropylcarbodiimide.

The molar dosage of the condensing reagent is 1.2-6 times total mole number of amino groups in an amino resin, preferably 2.5-3.5 times.

The activating reagent is selected from 1-hydroxybenzotriazole (HOBt) and N-hydroxy-7-aza-benzotriazole (HOAt), preferably HOBt.

The dosage of the activating reagent is 1.2-6 times total mole number of amino groups in the amino resin, preferably 2.5-3.5 times.

The coupling reaction time is 60-300 min, preferably 100-140 min

Preferably, the Fmoc-Leu-carrier resin has a substitution value of 0.5-1 5 mmol/g, and can present higher yield when the substitution value is preferably 0.8-1.2 mmol/g.

The carrier resin is a Trityl-Cl type resin or a hydroxyl resin. The Trityl-Cl type resin is preferably a Trityl-Cl resin, 4-Methyltrityl-Cl resin, 4-Methoxytrityl-Cl resin or 2-Cl Trity-Cl resin, and the hydroxyl resin is preferably a Wang resin or hydroxymethyl phenoxymethyl polystyrene (HMP) resin.

When the Trityl-Cl resin is employed as the carrier resin, the method of coupling Fmoc-Leu-OH with the carrier resin comprises coupling the protected amino acid by an esterification reaction between a carboxyl of Fmoc-Leu-OH and a Cl-alkyl of the resin in the presence of alkali.

The alkali is selected from at least one of N,N-Diisopropylethylamine (DIEA), triethylamine (TEA) and pyridine, preferably DIEA. The molar dosage of the alkali is 1.5-3 times of the mole number of the protected amino acid.

The esterification reaction time is 1-6 hrs, preferably 3 hrs.

When the hydroxyl resin is employed as the carrier resin, the method of coupling Fmoc-Leu-OH to the carrier resin comprises coupling the protected amino acid by an esterification reaction between a carboxyl of Fmoc-Leu-OH and a hydroxyl of the resin in the presence of a coupling agent, an activating agent, and a base catalyst.

The coupling agent is selected from at least one of N,N-diisopropylcarbodiimide (DIC), N,N-dicyclohexylcarbodiimide (DCC), benzotriazole-1-yl-oxy-tris-pyrrolidino-phosphonium hexafluorophosphate (PyBOP), 2-(7-aza-1H-benzotriazole-1-yl)-1,1,3,3-tetramethyluronium hexafluorophosphate (HATU), benzotriazole-N,N,N′,N′-tetramethyluroniumhexafluophosphate (HBTU) or O-benzotriazole-N,N,N′,N′-tetramethyluronium tetrafluobate (TBTU), preferably DIC. The dosage of the coupling agent is 1.2-6 times of total mole number of the fed resin, preferably 3 times.

The base catalyst is 4-N,N-dimethyl pyridine (DMAP), and the dosage is 0.1 time total mole number of the fed resin.

The activating agent is selected from at least one of 1-hydroxybenzotriazole (HOBt) and N-hydroxy-7-aza-benzotriazole (HOAt), preferably HOBt. The dosage of the activating agent is 1.2-6 times total mole number of the fed resin, preferably 3 times.

The esterification reaction time is 12-36 hrs, preferably 18 hrs.

Further, the crude bivalirudin represented by SEQ. ID NO. 1 is obtained by acidolysis of the bivalirudin resin to remove the resin and side chain protecting groups:

SEQ. ID NO. 1
D-Phe-Pro-Arg-Pro-X-Asn-Gly-Asp-Phe-
Glu-Glu-Ile-Pro-Glu-Glu-Tyr-Leu-OH

wherein X represents Gly-Gly-Gly-Gly.

An acidolysis reagent involved therein is a mixed solvent comprising trifluoroacetic acid (TFA), 1,2-ethanedithiol (EDT), and water, and dosage thereof is 4-15 mL for 1 g resin, and the acidolysis reagent comprises 80-95% (v/v) of TFA , 1-10% (v/v) of EDT, and the remainder is water, particularly, 89-91% (v/v) of TFA , 4-6% (v/v) of EDT, and the remainder is water, and more particularly, 90% (v/v) of TFA, 5% (v/v) of EDT, and the remainder is water.

In 1 g bivalirudin resin, the dosage of the acidolysis agent is required to be 4-15 mL, and preferably, 1 g bivalirudin resin requires 9-11 mL of the acidolysis agent.

The cracking time of the acidolysis agent is 1-5 hrs at room temperature, preferably 2 hrs.

Further, pure bivalirudin is obtained by purification of the crude bivalirudin by high performance liquid chromatography and lyophilization. The purification method is described as follows:

Crude bivalirudin powder is weighed, added to water (approximate 20 mL water/g crude bivalirudin) and stirred, then dilute ammonia is added dropwise to adjust pH to 4.5-5.5, and the resulting solution is filtered by 0.45 μm hybrid microporous filter membrane for subsequent use.

Chromatographic packing for purification by high performance liquid chromatography is 10 pm reversed phase C18, mobile phase is 0.1% TFA/aqueous solution-0.1% TFA/acetonitrile solution, flow rate of a 77 mm*250 mm chromatographic column is 90 mL/min, and a gradient system is used for elution and purification by cycle sample injection. Supernatant of the crude bivalirudin solution is added to the chromatographic column, the mobile phase is started for elution, then the main peak is collected and acetonitrile is removed by evaporation to obtain a purified bivalirudin intermediate concentrate.

The purified bivalirudin intermediate concentrate is filtered by 0.45 μm filter membrane for subsequent use. High performance liquid chromatography is used for salt exchange, the mobile phase system is 0.1% TFA/aqueous solution-acetonitrile, the chromatographic packing for purification is 10 pm reversed phase C18, and the flow rate of the 77 mm*250 mm chromatographic column is 90 mL/min (corresponding flow rate can be adjusted according to chromatographic columns of different specifications). Sample is injected into the chromatographic column by gradient elution and cycle sample injection, the mobile phase is started for elution, then chromatogram is collected to observe changes in absorbance, the salt exchange main peak is collected and the purity is determined by analytical liquid chromatography, the salt exchange main peak solution is combined for vacuum concentration in water bath below 40° C., and most of the acetonitrile is evaporated by a rotary evaporator to obtain bivalirudin trifluoroacetate aqueous solution which is lyophilized to obtain the product.

The following examples will be helpful for understanding the invention, but should not be construed as limit thereto.

### EXAMPLE 1

### Preparation of Fmoc-Leu-Wang Resin

First, 500 g of Wang resin (the substitution value thereof was 1 0 mmol/g) was swelled with 5 L of N,N-dimethylformamide (DMF) for 30 min, then 353 g (1.0 mol) of Fmoc-Leu-OH was added and stirred for 30 min, 155 mL of DIC (1.0 mol), 135 g of HOBt (1.0 mol) and 6.1 g (0.05 mol) of DMAP were added, stirred at room temperature for reaction for 18 hrs, then the resin was washed respectively with DMF, dichloromethane (DCM) and methanol for three times after filtration, and dried under vacuum to obtain 651 g of Fmoc-Leu-Wang resin, with the esterification yield of 95.6%.

### EXAMPLE 2

### Preparation of H-Leu-Wang Resin by Fmoc Deprotection of Fmoc-Leu-Wang Resin

The Fmoc-Leu-Wang resin was swelled with 5 L of 20% piperidine (PIP)/NN-dimethylformamide (DMF) solution for 10 min, then 5 L of 20% PIP/DMF solution was added after filtration and stirred at room temperature for reaction for 25 min, then the resin was washed respectively with DMF, DCM and methanol for three times after filtration, and dried under vacuum to prepare H-Leu-Wang resin.

### EXAMPLE 3

### Preparation of Fmoc-Leu-2-Cl-Trt Resin

First, 500 g of 2-Cl-Trt-Cl resin (the substitution value was 1 0 mmol/g) was swelled with 5 L of N,N-dimethylformamide (DMF) for 30 min, 353 g (1.0 mol) of Fmoc-Leu-OH was added and stirred for 30 min, then 260 mL of DIEA (1.5mol) was added and stirred at room temperature for reaction for 3 hrs, the resin was washed respectively with DMF, DCM and methanol for three times after filtration, and dried under vacuum to obtain 655 g of Fmoc-Leu-2-Cl-Trt resin, with the esterification yield of 98.1%.

### EXAMPLE 4

### Preparation of H-Leu-2-Cl-Trt Resin by Fmoc Deprotection of Fmoc-Leu-2-Cl-Trt Resin

The Fmoc-Leu-2-Cl-Trt resin was swelled with 5 L of 20% PIP/DMF solution for 10 min, 5 L of 20% PIP/DMF solution was added after filtration and stirred at room temperature for reaction for 25 min, then the resin was washed respectively with DMF, DCM and methanol for three times after filtration, and dried under vacuum to prepare H-Leu-2-Cl-Trt resin.

### EXAMPLE 5

### Synthesis of Fmoc-Gly-Gly-Gly-Gly-OH

First, 3.0 mol of Fmoc-Gly and 3.0 mol of HOBt were dissolved with proper amount of DMF; then another 3.0 mol of DIC was slowly added to protected amino acid DMF solution while stirring, and stirred at room temperature for reaction for 30 min to obtain activated protected amino acid solution.

First, 1 kg of Fmoc-Gly-2-Cl-Trt-resin (the substitution value was 1.0 mmol/g) was subject to Fmoc deprotection using 5 L of 20% PIP/DMF solution for 25 min, then the resin was washed respectively with MDF and DCM for three times after filtration, the protected amino acid solution was added and stirred at room temperature for reaction for 3 hrs, then the resin was washed respectively with MDF and DCM for three times after filtration upon completion of the reaction.

The two reaction steps were repeated, and another 3 Glys were coupled to prepare a Fmoc-Gly-Gly-Gly-Gly-2-Cl-Trt-resin.

The Fmoc-Gly-Gly-Gly-Gly-2-Cl-Trt-resin was added to 20 L of 30% hexafluoroisopropanol/DCM solution and stirred for reaction for 2 hrs, then the filtrate was collected after filtration, and the solvent was dried by distillation under vacuum to obtain 457 g of Fmoc-Gly-Gly-Gly-Gly-OH, with the yield of 97.2%, the purity of 98.3%, and MS m/z of 469 (M+1).

### EXAMPLE 6

### Synthesis of Bivalirudin Resin

The bivalirudin resin was represented by SEQ. ID NO. 3:

SEQ. ID NO. 3
H-D-Phe-Pro-Arg(Pbf)-Pro-X-Asn(Trt)-Gly-Asp(OtBu)-
Phe-Glu(OtBu)-Glu(OtBu)-Ile-Pro-Glu(OtBu)-
Glu(OtBu)-Tyr(tBu)-Leu-resin

wherein X represented Gly-Gly-Gly-Gly.

Fmoc-Leu-Wang resin was successively coupled with the protected amino acids shown in Table 1 to obtain a bivalirudin resin. The protected amino acids corresponding to the 2^{nd} to 17^{th} amino acids from the resin of the protected amino acids used in the example are as follows:

TABLE 1

| Peptide | | |
|---|---|---|
| coupling sequence n = | Protected amino acids | Molecular weight |
| 2 | Fmoc-Tyr(tBu)—OH | 460 |
| 3 | Fmoc-Glu(OtBu)—OH | 426 |
| 4 | Fmoc-Glu(OtBu)—OH | 426 |
| 5 | Fmoc-Pro-OH | 337 |
| 6 | Fmoc-Ile-OH | 353 |
| 7 | Fmoc-Glu(OtBu)—OH | 426 |
| 8 | Fmoc-Glu(OtBu)—OH | 426 |
| 9 | Fmoc-Phe-OH | 387 |
| 10 | Fmoc-Asp(OtBu)—OH | 412 |
| 11 | Fmoc-Gly-OH | 297 |
| 12 | Fmoc-Asn(Trt)-OH | 597 |
| 13 | Fmoc-Gly-Gly-Gly-Gly-OH | 468 |
| 14 | Fmoc-Pro-OH | 337 |
| 15 | Fmoc-Arg(pbf)-OH | 648 |
| 16 | Fmoc-Pro-OH | 337 |
| 17 | Fmoc-D-Phe-OH | 387 |

The 13^{th} protected amino acid is Fmoc-Gly-Gly-Gly-Gly-OH prepared in Example 5.

The activation method of the protected amino acid comprises the following steps:

First, 1.5 mol of protected amino acid and 1.5 mol of HOBt were dissolved with proper amount of DMF; another 1.5 mol of DIC was slowly added to protected amino acid DMF solution while stirring, and stirred at room temperature for reaction for 30 min to obtain activated protected amino acid solution.

Then 0.5 Kg of Fmoc-Leu-Wang resin (the substitution value was 1 0 mmol/g) was subject to deprotection using 5 L of 20% PIP/DMF for 25 min, and filtered to obtain the Fmoc-deprotected resin for subsequent use.

The Fmoc-deprotected resin was added to the second activated protected amino acid solution for coupling reaction for 60-300 min, then filtered and washed to obtain 2-peptide resin. The 2-peptide resin was subject to Fmoc deprotection using 5 L of 20% PIP/DMF solution for 25 min, filtered and washed for coupling reaction with the third activated protected amino acid solution for 60-300 min, and then filtered and washed to obtain 3-peptide resin.

The Fmoc-protected amino acids corresponding to the 4^{th} to 17^{th} amino acids (i.e. the Fmoc-[1-(n−1)^{th}]amino acid-Wang resin obtained in the previous step) were successively coupled by the same method for coupling reaction with the activated Fmoc-protected amino acid (n^{th}) for 60-300 min after Fmoc deprotection, with n=217. After all protected amino acids were coupled, 5 L 20% PIP/DMF solution was used for Fmoc deprotection for 25 min, then filtered and washed to obtain the bivalirudin resin.

### EXAMPLE 7

### Acidolysis of Bivalirudin Resin

The bivalirudin resin prepared in Example 6 was mixed with a cracking reagent [TFA/water/EDT=95:5:5 (V/V) (10 mL/g resin), and evenly stirred at room temperature for reaction for 3 h, then a sand core funnel was used for filtering the reaction mixture, and the filtrate was collected, then the resin was washed with small amount of TFA for three times, the filtrates were combined and concentrated under vacuum, anhydrous ether was added for precipitation, and the precipitate was washed with anhydrous ether for three times, and dried to obtain white powder that is crude bivalirudin.

### EXAMPLE 8

### Purification of Crude Bivalirudin

Crude bivalirudin powder was weighed, added to purified water (approximately 20 mL of water/g crude bivalirudin), then dilute ammonia was added dropwise while stirring to adjust pH to about 5.0, and the resulting solution was filtered by 0.45 μm of hybrid microporous filter membrane for purification.

High performance liquid chromatography was used for purification, the chromatographic packing for purification was 10 μm of reversed phase C18, the mobile phase system was 0.1% TFA/aqueous solution-0.1% TFA/acetonitrile solution, the flow rate of a 77 mm*250 mm chromatographic column was 90 mL/min, a gradient system was used for elution and purification by cycle sample injection. Supernatant of the crude bivalirudin solution was added to the chromatographic column, the mobile phase was started for elution, then the main peak was collected and acetonitrile was removed by evaporation to obtain a purified bivalirudin intermediate concentrate.

The purified bivalirudin intermediate concentrate was filtered by 0.45 μm filter membrane for subsequent use. High performance liquid chromatography was used for salt exchange, the mobile phase system was 0.1% TFA/aqueous solution-acetonitrile, the chromatographic packing for purification was 10 μm reversed phase C18, and the flow rate of the 77 mm*250 mm chromatographic column was 90 mL/min (corresponding flow rate can be adjusted according to chromatographic columns of different specifications). Sample was injected into the chromatographic column by gradient elution and cycle sample injection, the mobile phase was started for elution, then the chromatogram was collected to observe changes in absorbance, the salt exchange main peak was collected and the purity was determined by analytical liquid chromatography, the salt exchange main peak solution was combined for vacuum concentration in water bath below 40° C., and most of the acetonitrile was evaporated by a rotary evaporator to obtain bivalirudin trifluoroacetate aqueous solution which is lyophilized to obtain 608 g product, with the total yield of 55.8%. Molecular weight: 2181.2 (100% M+H); specific rotation: −116.5°; Moisture: 2.1%, trifluoracetic acid: 9.5%; purity: 99.8%.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A method for preparing bivalirudin, the method comprising preparing a bivalirudin resin by a solid phase synthesis method, performing acidolysis of the bivalirudin resin to obtain crude bivalirudin, and purifying the crude bivalirudin to obtain purified bivalirudin, wherein the solid phase synthesis method comprises successively coupling Fmoc-protected amino acids corresponding to a sequence represented by SEQ. ID NO. 2 on an Fmoc-Leu-carrier resin through a solid phase coupling synthesis method to obtain the bivalirudin resin represented by SEQ. ID NO. 2:
SEQ. ID NO. 2
R₁-D-Phe-Pro-Arg(Pbf)-Pro-X-Asn(R₂)-Gly-Asp(OtBu)-
Phe-Glu(OtBu)-Glu(OtBu)-Ile-Pro-Glu(OtBu)-
Glu(OtBu)-Tyr(tBu)-Leu-resin
wherein X represents Gly-Gly-Gly-Gly, R₁ represents R₃ or H, R₂ represents Trt or H, and R₃ represents Fmoc; and a solid phase coupling synthetic reaction for coupling an X fragment is carried out only once.

2. The method of claim 1, wherein a substitution value of the Fmoc-Leu-carrier resin is between 0.5 and 1.5 mmol/g.

3. The method of claim 2, wherein the substitution value of the Fmoc-Leu-carrier resin is between 0.8 and 1.2 mmol/g.

4. The method of claim 1, wherein the Fmoc-Leu-carrier resin is a Trityl-Cl type resin or a hydroxyl resin.

5. The method of claim 4, wherein the Trityl-Cl type resin is a Trityl-Cl resin, 4-Methyltrityl-Cl resin, 4-Methoxytrityl-Cl resin, or 2-Cl Trity-Cl resin, and the hydroxyl resin is a Wang resin or hydroxymethyl phenoxymethyl polystyrene resin.

6. The method of claim 5, wherein when the Trityl-Cl resin is employed as the Fmoc-Leu-carrier resin, a step for coupling Fmoc-Leu-OH and the Fmoc-Leu-carrier resin comprises coupling a protected amino acid by an esterification reaction between a carboxyl of Fmoc-Leu-OH and a Cl-alkyl of the Fmoc-Leu-carrier resin in the presence of alkali.

7. The method of claim 6, wherein the alkali is selected from at least one of N,N-Diisopropylethylamine, triethylamine, and pyridine.

8. The method of claim 5, wherein when the hydroxyl resin is employed as the Fmoc-Leu-carrier resin, a step for coupling Fmoc-Leu-OH and the Fmoc-Leu-carrier resin comprises coupling a protected amino acid by an esterification reaction between a carboxyl of Fmoc-Leu-OH and a hydroxyl of the Fmoc-Leu-carrier resin in the presence of a coupling agent, an activating agent, and a base catalyst.

9. The method of claim 8, wherein the coupling agent is selected from at least one of N,N-diisopropylcarbodiimide, N,N-dicyclohexylcarbodiimide, benzotriazole-1-yl-oxy-tris-pyrrolidino-phosphonium hexafluorophosphate, 2-(7-aza-1H-benzotriazole-1-yl)-1,1,3,3-tetramethyluronium hexafluorophosphate, benzotriazole-N,N,N′,N′-tetramethyluroniumhexafluophosphate, and O-benzotriazole-N,N,N′,N′-tetramethyluronium tetrafluobate.

10. The method of claim 8, wherein the base catalyst is 4-N,N-dimethyl pyridine.

11. The method of claim 8, wherein the activating agent is selected from at least one of 1-hydroxybenzotriazole and N-hydroxy-7-aza-benzotriazole.

12. The method of claim 1, wherein the Fmoc-protected amino acids comprise R₃-D-Phe-OH, Fmoc-X—OH, Fmoc-Asn(R₂)-OH, Fmoc-Arg(pbf)-OH, Fmoc-Asp (OtBu)-OH, Fmoc-Glu(OtBu)-OH, Fmoc-Gly-OH, Fmoc-Ile-OH, Fmoc-Leu-OH, Fmoc-Phe-OH, Fmoc-Pro-OH and Fmoc-Tyr(tBu)-OH respectively, wherein X represents Gly-Gly-Gly-Gly, R₂ represents Trt or H, and R₃ represents Fmoc.

13. The method of claim 7, wherein the Fmoc-protected amino acids comprise R₃-D-Phe-OH, Fmoc-X—OH, Fmoc-Asn(R₂)-OH, Fmoc-Arg(pbf)-OH, Fmoc-Asp (OtBu)-OH, Fmoc-Glu(OtBu)-OH, Fmoc-Gly-OH, Fmoc-Ile-OH, Fmoc-Leu-OH, Fmoc-Phe-OH, Fmoc-Pro-OH and Fmoc-Tyr(tBu)-OH respectively, wherein X represents Gly-Gly-Gly-Gly, R₂ represents Trt or H, and R₃ represents Fmoc.

14. The method of claim 11, wherein the Fmoc-protected amino acids comprise R₃-D-Phe-OH, Fmoc-X—OH, Fmoc-Asn(R₂)-OH, Fmoc-Arg(pbf)-OH, Fmoc-Asp (OtBu)-OH, Fmoc-Glu(OtBu)-OH, Fmoc-Gly-OH, Fmoc-Ile-OH, Fmoc-Leu-OH, Fmoc-Phe-OH, Fmoc-Pro-OH and Fmoc-Tyr(tBu)-OH respectively, wherein X represents Gly-Gly-Gly-Gly, R₂ represents Trt or H, and R₃ represents Fmoc.

15. The method of claim 1, wherein the crude bivalirudin represented by SEQ. ID NO. 1 is obtained by acidolysis of the bivalirudin resin to remove the Fmoc-Leu-carrier resin and side chain protecting groups:
SEQ. ID NO. 1
D-Phe-Pro-Arg-Pro-X-Asn-Gly-Asp-Phe-
Glu-Glu-Ile-Pro-Glu-Glu-Tyr-Leu-OH
wherein X represents Gly-Gly-Gly-Gly; an acidolysis reagent involved therein is a mixed solvent comprising trifluoroacetic acid (TFA), 1,2- ethanedithiol (EDT), and water, and dosage thereof is 4-15 mL for 1 g resin, and the acidolysis reagent comprises 80-95% (v/v) of TFA , 1-10% (v/v) of EDT, and the remainder is water.
